(19) United States
(12) Patent Application Publication
(10) Pub. No.: US 2024/0307231 A1
(43) Pub. Date: Sep. 19, 2024

(54) TYMPANOSTOMY TUBE INSERTION DEVICE

(71) Applicant: Ear Tech, LLC, Indianapolis, IN (US)

(72) Inventor: Michael H Fritsch, Indianapolis, IN (US)

(73) Assignee: Ear Tech, LLC, Indianapolis, IN (US)

(21) Appl. No.: 18/603,173

(22) Filed: Mar. 12, 2024

Related U.S. Application Data
(60) Provisional application No. 63/451,906, filed on Mar. 13, 2023.

(51) Int. Cl. A61F 11/20

(52) U.S. Cl. CPC A61F 11/202

(57) ABSTRACT

A tympanostomy tube insertion system includes a guide tube including a hollow passageway, the guide tube extending between a proximal end and a distal end. A first cutting member is located at the distal end of the guide tube. A plunger member has a forward end which extends into the hollow passageway at a proximal end of the guide tube. A tympanostomy tube is disposed within the hollow passageway toward the distal end of the guide tube.

### TECHNICAL FIELD OF THE INVENTION

The present application relates to medical devices, and more specifically, but not exclusively, to an insertion system for a tympanostomy tube.

### BACKGROUND

From time to time, most younger children suffer from earaches. In many cases, an earache is caused by a buildup of fluid in the middle ear (tympanic cavity) that leads to an infection in the ear. Usually, the earache can be treated by giving the child an antibiotic that will help to treat this middle ear infection.

Unfortunately, antibiotics do not work well with all patients, for although the antibiotic helps to cure the infection, some patients accumulate fluid frequently enough within their middle ears so that it is necessary to take steps to aerate the middle ear to thereby help prevent the accumulation of fluid. This aeration helps to reduce the fluid buildup and thereby reduce the likelihood that bacteria will cause an infection in the accumulated fluid, which thereby helps to reduce or eliminate the recurrence of earaches.

To treat such patients, a tympanostomy tube is often inserted into the eardrum (tympanic membrane) to extend through the eardrum in order to keep the middle ear aerated for a prolonged period of time, and to prevent the accumulation of fluid in the inner ear. A tympanostomy tube is also known as a grommet, ear tube, pressure equalization tube, PE tube, or a myringotomy tube.

The operation to insert the tube is referred to as a myringotomy (tympanostomy) and is performed under local or general anesthesia. A myringotomy is a surgical procedure in which a tiny incision is created in the eardrum, so as to relieve pressure caused by the excessive buildup of fluid, or to drain puss, and wherein a tube is inserted in the eardrum for residence over an extended period of time.

The most commonly used type of ear tube is shaped like a grommet. If a medical practitioner decides that the ear needs to be kept open and ventilated for a long period of time, a “T” shaped tube may be used, as these “T” tubes can stay in place two to four years or so. The materials of choice for creating such tubes are plastic materials such as silicone or Teflon. Formerly, stainless steel tubes and other materials were popular, but they are no longer used frequently.

The placement of ear tubes in a child's ear is a very common procedure. In the U.S., it is estimated that the most common reason for a child to undergo a general anesthetic is the insertion of such ear tubes within the child's ear. Ear tubes (tympanostomy tubes) generally remain within the eardrum for an extended period of time, usually lasting between six months and two years, although “T” tubes can last for four years or longer. Ear tubes generally spontaneously fall out of the eardrum as the skin of the eardrum slowly migrates out toward the ear canal wall over time. The ear drum usually closes without a residual hole at the tube site, but in a small number of cases, a perforation can exist.

In the conventional manner for performing tube insertion, the first step is to make a myringotomy incision by inserting a needle-like knife into the ear canal to make the incision. After the incision is made, the grommet-shaped ear tube is then grasped with forceps and half of the grommet is inserted through the incision to finally rest suspended within the eardrum, so that the passageway in the grommet creates an air passage between the auditory canal and tympanic cavity.

A typical ear tube grommet is shaped similarly to a thread spool or wire spool. The grommet generally includes a reduced diameter central portion having a cylindrical radially outwardly facing surface. A first relatively enlarged diameter flange having a cylindrical radially outwardly facing perimetral edge is placed at one end of the reduced diameter portion, and a second, similarly configured enlarged diameter portion is placed at the second end of the reduced diameter portion. An axially extending air passageway extends between a first end and a second end of the spool. The first and second end flanges also include generally planar upper and lower surfaces that have a generally round shape.

When inserted in the eardrum, the first enlarged diameter portion is disposed externally of the eardrum, with the second enlarged diameter portion disposed interiorly of the eardrum. The reduced diameter central portion extends through the eardrum. The result is that the first and second enlarged diameter portions prevent the grommet-shaped tube from becoming disconnected from the eardrum, to thus hold the grommet so that it is suspended within its position within the eardrum. When held in the proper position, the axially extending passageway of the tube can pass between the inner and outer surfaces of the eardrum, to thereby provide aeration to the middle ear, which comprises that portion of the ear that is disposed just interiorly of the eardrum.

Although such ear tubes and insertion devices serve their intended purposes well, room for improvement exists. In particular, the generally small size of an ear tube makes it very difficult and tricky to manipulate the tube properly to insert it into the eardrum. In particular, it is difficult for even skilled surgeons to line up the grommet properly to insert it into the very tiny incision that was recently made in the eardrum by the knife. In essence, the doctor must move the knife into and out of the ear to make the incision, and then follow it up with an insertion of the grommet into the ear, within the same incision that was just made by the knife.

Problems can also arise after insertion of the tympanostomy tube. If the tympanic cavity of the patient has very viscous fluid, such as thick mucus, the surgeon may be unable to successfully suction the viscous fluid through the air passageway of the tympanostomy tube.

Therefore, further technological developments are desirable.

### SUMMARY

One form of the present application includes a unique tympanostomy tube insertion system. Other forms include unique tympanostomy tube apparatuses, devices, systems, and methods. Further embodiments, inventions, forms, objects, features, advantages, aspects, and benefits of the present application are otherwise set forth or become apparent from the description and drawings included herein.

### BRIEF DESCRIPTION OF THE DRAWINGS

The description herein makes reference to the accompanying drawings wherein like reference numerals refer to like parts throughout the several views, and wherein:

FIG. 1 is a side, partly schematic view of a prior art “T” type tympanostomy tube in its maintenance position;

FIG. 2 is a side view of the prior art “T” type tympanostomy tube of FIG. 1 in its insertion position;

FIG. 3 is a side view of the “T” type tympanostomy tube of the present invention, shown in its maintenance position;

FIG. 4 is a side view of the inventive tympanostomy tube of FIG. 3, shown in its insertion position;

FIG. 5 is a sectional view taken along lines 5-5 of FIG. 4;

FIG. 6 is a side, partly sectional view of the “T” type tympanostomy tube and insertion tools inserted in an ear canal, and just prior to the insertion of the tympanostomy tube in the tympanic membrane (eardrum);

FIG. 7 is a side, partly sectional, progressive view, showing the tympanostomy tube as it is being inserted into and extending through the tympanic membrane;

FIG. 8 is a side view of the “T” type tympanostomy tube and insertion tool of the present invention, showing the tympanostomy tube fully inserted into the tympanic membrane with the tympanostomy tube shown in its maintenance position;

FIG. 9 is a side, partly sectional view showing the tympanostomy tube fully inserted into the eardrum and in the maintenance position, with the plunger being removed from the insertion tube;

FIG. 10 is a sectional view showing the “T” type tympanostomy tube fully inserted in the tympanic membrane, with the tympanostomy tube in its maintenance position, and the insertion tube and plunger removed;

FIG. 11 is a side, partly sectional view of an alternative tympanostomy tube insertion system, according to a further form of the present application, which depicts an exemplary guide tube cutting member extending outwardly from a distal end of the guide tube;

FIG. 12 is a perspective side view focused upon the distal end of the guide tube of FIG. 11, with the tympanostomy tube removed from the guide tube for clarity, which depicts an exemplary bladed guide tube cutting member;

FIG. 13A is a perspective side view focused upon the distal end of the guide tube, with the tympanostomy tube removed from the guide tube for clarity, which depicts an exemplary serrated guide tube cutting member;

FIG. 13B is a perspective side view of an alternative serrated guide tube cutting member;

FIG. 14 is a perspective side view focused upon the distal end of the guide tube, with the tympanostomy tube removed from the guide tube for clarity, which depicts an exemplary scalpel type guide tube cutting member;

FIG. 15 is a side, partly sectional, progressive view, showing the guide tube cutting member of FIG. 11 incising the tympanic membrane;

FIG. 16 is a side, partly sectional, depicting the tympanostomy tube insertion system pulled slightly away from the tympanic membrane after the guide tube cutting member has incised the tympanic membrane, depicting the tympanostomy tube within the guide tube ready for insertion into the tympanic membrane;

FIG. 17 is a side, partly sectional, progressive view, showing an exemplary tympanostomy tube being inserted into and extending through the tympanic membrane;

FIG. 18 is a side, partly sectional view depicting the tympanostomy tube fully inserted into the tympanic membrane with the tympanostomy tube shown in its maintenance position;

FIG. 19A-19C are side, partly sectional, progressive views depicting the tympanostomy tube insertion system of FIG. 11 utilized in a one-step fashion, depicting the guide tube rotated ninety degrees so that the cutting member is at an upper position for clarity;

FIG. 20 depicts a tympanic membrane with a tympanostomy tube inserted therein, which further depicts suction being performed on the tympanic cavity through an aperture created by the guide tube cutting member;

FIG. 21 is a diagrammatic view of the anatomy of the ear of a human being;

FIG. 22 is a side view of a first alternate embodiment “T” type tympanostomy tube of the present invention having a serrated cutting surface for helping to reduce tearing of the tympanic membrane when inserted therethrough;

FIG. 23 is a sectional view taken along lines 23-23 of FIG. 22;

FIG. 24 is a sectional view taken along lines 24-24 of FIG. 22;

FIG. 25 is a greatly enlarged, sectional view taken generally along lines 25-25 of FIG. 24;

FIG. 26 is a second alternate embodiment “T” type tympanostomy tube that also includes a serrated cutting surface to reduce tearing of the tympanic membrane when inserted therethrough;

FIG. 27 is a sectional view taken generally along lines 27-27 of FIG. 26;

FIG. 28 is a sectional view taken generally along lines 28-28 of FIG. 26;

FIG. 28A is a greatly enlarged, sectional view taken generally along lines 28A-28A of FIG. 28;

FIG. 29 is a front side view of a third alternate embodiment “T” type tympanostomy tube of the present invention having a serrated cutting edge to facilitate the cutting, and reduce the tearing of the tympanic membrane as the tympanostomy tube is inserted therethrough;

FIG. 30 is a side view of the third alternate embodiment tympanostomy tube shown in FIG. 29;

FIG. 31 is a sectional view taken generally along lines 31-31 of FIG. 30;

FIG. 32 is a sectional view taken generally along lines 32-32 of FIG. 30;

FIG. 33 is a side view of a serrated edged T-tube having solid legs shown disposed in the insertion or non-splayed position;

FIG. 34 is a side view of the T-tube of FIG. 33, wherein the legs are shown in their splayed, post-installation maintenance position;

FIG. 35 is a side view of an alternate embodiment T-tube, wherein the legs are generally hollow, and the end is serrated;

FIG. 36 is a side view of the T-tube shown in FIG. 35, wherein the legs are shown in their splayed position, as they would be when inserted into an ear of a patient through the ear drum;

FIG. 37 is a side view of an alternate embodiment bi-pod T-tube of the present invention;

FIG. 38 is a side view of the alternate embodiment of FIG. 37, wherein the legs are shown in the maintenance position;

FIG. 39 is a side view of a quad leg embodiment T-tube tympanostomy tube;

FIG. 40 is an enlarged sectional view taken along lines 40-40 of FIG. 39;

FIG. 41 is a top view of the quad legged tympanostomy tube shown with the four legs in the splayed or maintenance position;

FIG. 42 is a sectional view taken generally along lines 42-42 of FIG. 41;

FIG. 43 is a top view of the quad tube of FIG. 40 wherein the legs are shown in their splayed or installed position;

FIG. 44 is a side view of another embodiment of a quad-like T tube of the present invention, wherein upstanding blades having serrated or toothed surfaces are formed;

FIG. 45 is an enlarged, end view of the embodiment shown in FIG. 44;

FIG. 46 is a side view of the embodiment of FIG. 44, showing the legs in the splayed, or maintenance position;

FIG. 47 is an enlarged, sectional view, taken along lines 4-75-4-75 of FIG. 4-64; and

FIG. 48 is a top view of the embodiment shown in FIG. 44, wherein the legs are in their splayed position.

### DETAILED DESCRIPTION OF INVENTION

For purposes of promoting an understanding of the principles of the invention, reference will now be made to the embodiments illustrated in the drawings and specific language will be used to describe the same. It will nevertheless be understood that no limitation of the scope of the invention is thereby intended, any alterations and further modifications in the illustrated device, and any further applications of the principles of the invention as illustrated therein being contemplated as would normally occur to one skilled in the art to which the invention relates.

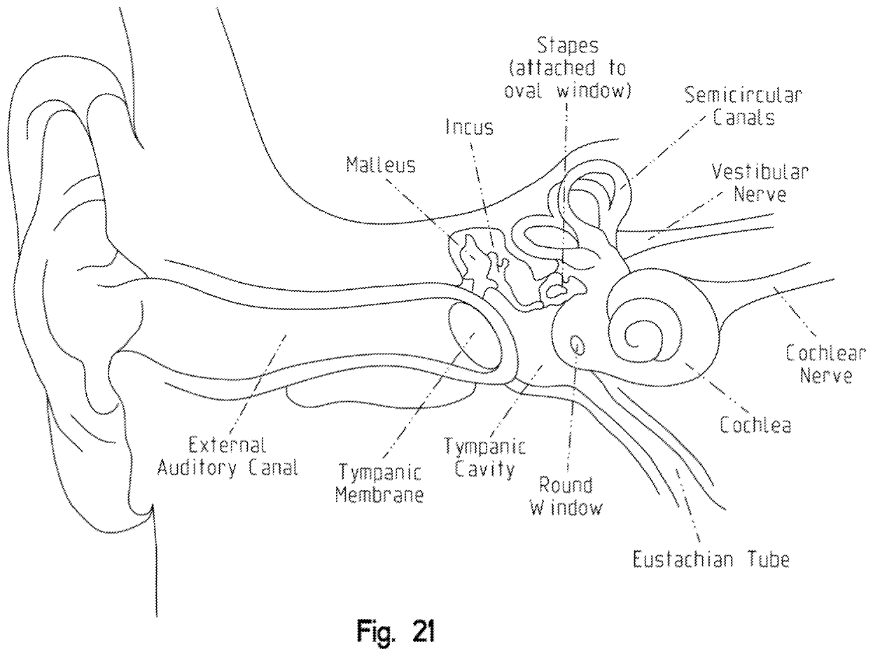

Turning first to FIG. 21, a diagrammatic representation of the ear and its component parts is shown. This drawing is provided for reference to help provide context for the description of the tympanic tube of the present invention, and its placement within the ear.

The tympanic tube is inserted through the external auditory canal. The tube is positioned adjacent to the lateral (exterior) surface of the tympanic membrane, and then extended through the tympanic membrane into the tympanic cavity. The tympanic cavity is also known as the middle ear. When fully inserted and resident in the tympanic membrane, the tympanic tube will include a distal portion that is disposed adjacent to the medial (interior) surface of the tympanic membrane, and a proximal end that will be disposed adjacent to the exterior surface of the tympanic membrane and reside in the external auditory canal.

The tympanostomy tube also includes a generally cylindrical central portion that extends through the tympanic membrane, to couple the distal and proximal ends of the tympanostomy tube. When so inserted, the tympanostomy tube of the present invention provides for aeration of the middle ear by providing a venting passageway between the external auditory canal and the tympanic cavity. This aeration helps to reduce the amount of fluid buildup in the tympanic cavity, which thereby helps to reduce the likelihood of an infection occurring in the tympanic cavity. As infections in the middle ear often result in earaches to the patient, reducing the severity and/or number of middle ear infections leads to a reduction in earaches for the patient.

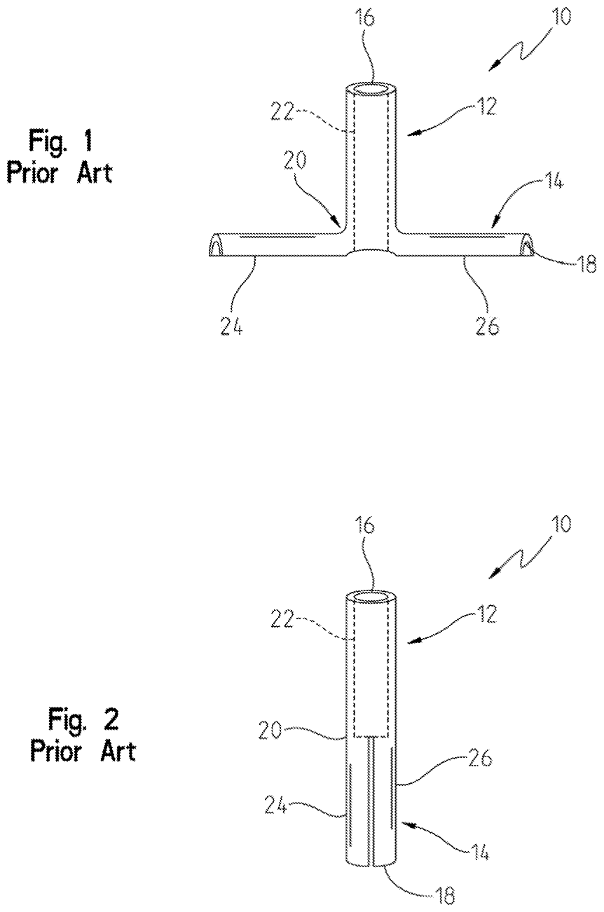

A prior known tympanostomy tube is shown in FIGS. 1 and 2. The prior tympanostomy tube 10 includes a proximal portion 12, and a distal portion 14. The tube 10 also includes a proximal end 16 that is located at the proximal end of the proximal portion 12, and a distal end 18 that is located at the distal end of the distal portion 14. A central portion 20 comprises the area of the tube 10 where the proximal portion 12 joins the distal portion 14. An axially-extending passageway 22 extends through the proximal portion 12, and terminates at an open end in the central portion 20. When the tube 10 is in its insertion position, as shown in FIG. 2, the axially-extending passageway 22 extends all the way between the proximal end 16 and the distal end 18. The distal portion 14 includes a first axially extending distal leg member 24, and a second axially extending distal leg member 26.

When in the insertion position, as shown in FIG. 2, it will be noted that the first and second distal legs 24, 26 are disposed generally co-axially with the proximal portion 12, and that they have the same general dispositions and configurations as the proximal portion 12. However, the distal legs 24, 26 are moveable between an insertion position 14, as shown in FIG. 2, and a maintenance position as shown in FIG. 1. When in the maintenance position, the distal legs 24, 26 are disposed at generally an oblique angle, and preferably perpendicular to the axis of the axially-extending passageway 22 that extends through the proximal portion 12.

The insertion of the prior art tube 10 occurs by first employing a knife to make an incision in the tympanic membrane. The prior art tube 10 is then inserted through the freshly cut incision within the tympanic membrane to a point wherein the distal legs 24, 26 of the distal portion 14 are fully inserted into the tympanic cavity. As discussed above, this insertion procedure is a very tricky two-step procedure requiring the insertion and removal of the knife, that is followed by the insertion instrument that is used to grip and manipulate the tube, into the tympanic membrane. Once the tube 10 is successfully inserted, the instrument is removed from the ear canal.

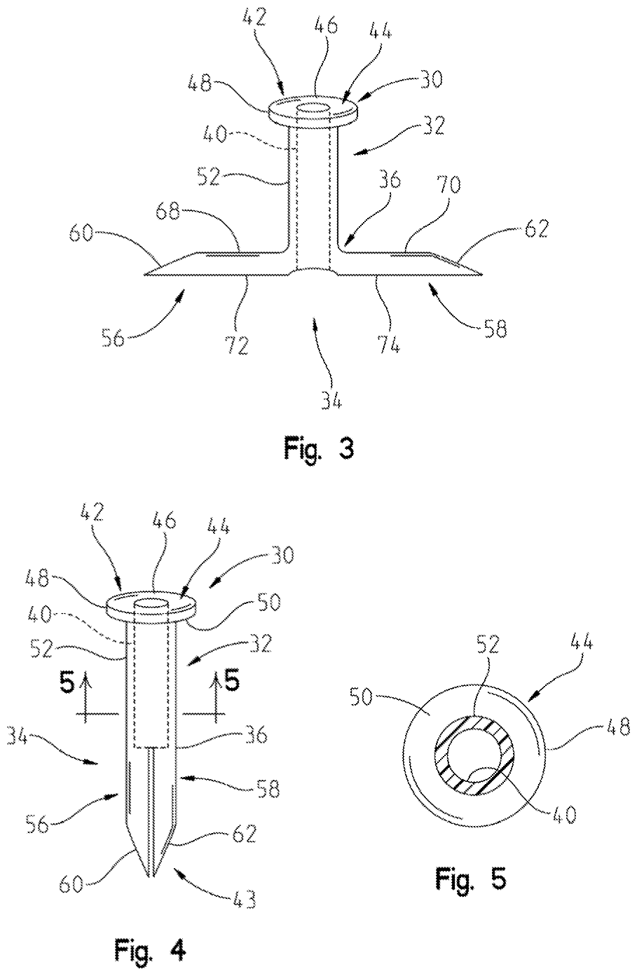

A first embodiment “T” type tympanostomy tube 30 of the present invention is shown in FIGS. 3-10. The tympanostomy tube 30 includes a proximal portion 32, a distal portion 34, and a central portion 36. The central portion 36 comprises that portion wherein the distal portion 34 joins the proximal portion 32.

An axially-extending passageway 40 extends axially through the proximal portion 32. When the tube 30 is in its insertion position, as shown in FIG. 4, the axially-extending passageway 40 also extends through the distal portion 34. The tube 30 also includes a proximal end 42 that is disposed at the proximal end of the proximal portion 32, and a distal end 43 that is disposed at the distal end of the distal portion 34.

A radially outwardly-extending flange 44 is formed at the proximal end 42 of the ear tube 30. The radially outwardly-extending flange 44 includes a proximally-facing end surface 46, and a radially outwardly facing perimetral edge 48. An axially distally-facing surface 50 is disposed in an opposed relationship to the proximally-facing end surface 46.

The radially extending flange 44 is designed to have a diameter larger than the incision made by the tympanostomy tube 30. The purpose of this larger diameter is to ensure that the tympanostomy tube 30 remains in its appropriate place on the tympanic membrane after insertion. The relatively enlarged diameter flange 44 helps to ensure that the tympanostomy tube 30 is not moved medially out of its engagement with the incision in the tympanic membrane, and through the tympanic membrane into the tympanic cavity.

The proximal portion 32 includes an axially-extending radially outwardly-facing generally cylindrical surface 52, which extends generally from the proximal end to the distal end of the proximal portion 32.

The distal portion 34 includes at least two distally disposed legs including a first distal leg 56 and a second distal leg 58. The first and second distal legs 56, 58 include, respectively, first and second distal ends 60, 62. The first and second distal ends 60, 62 are beveled or otherwise configured to have knifelike sharp edge surfaces, that comprise cutting surfaces. The first and second distal legs 56, 58 should have distal ends 60, 62 that are designed to be sharp enough to easily penetrate the tympanic membrane 98, upon the exertion of an axially and medially directed force on the tympanostomy tube 30, such as an axially-directed force that is applied to the proximally-facing surface 46 of the proximal flange 44 in a manner to move the tube 30 medially toward the tympanic cavity.

By employing cutting edge containing distal ends 60, 62, the need for using a knife to make a separate incision is thereby obviated. The insertion goes from two steps (i.e., (1) an incision followed by (2) the tube insertion, to one step (i.e., incise and insert, all in one). As best shown in FIGS. 3 and 4, the distal legs 56, 58 are moveable between an insertion position (FIG. 4) and a maintenance position (FIG. 3). In the insertion position (FIG. 4), the distal legs 56, 58 assume a position wherein they are disposed generally co-axially to the long axis of the passageway 40. In the insertion position, the cutting edges 60, 62 are positioned to cut into a membrane, such as the tympanic membrane, upon an axially, medially exerted force upon the tympanostomy tube 30.

From the insertion position (FIG. 4) the distal legs 56, 58 can be moved into a maintenance position as shown in FIG. 3. In the maintenance position, the long axes of each of the first and second distal legs 56, 58 are disposed at least at an oblique angle to the long axis of the passageway 40. Preferably, as shown in FIG. 3, the first and second legs 56, 58 are disposed generally co-axially to each other, and along an axis that is generally perpendicular to the long axis of the passageway 40.

When in the maintenance position as shown in FIG. 3, the distal legs 56, 58 each include laterally (exteriorly) facing surfaces 68, 70 and medially (interiorly) facing surfaces 72, 74. The designations “medial” and “lateral” are used to describe these surfaces because, when in the maintenance position and inserted into an ear, the laterally-facing surfaces 68, 70 face laterally, and are disposed against the medially-facing surface of the eardrum 98. The medially-facing surfaces 72, 74 face medially inwardly in the middle ear. It will be noted that the medial/lateral designations do not necessarily apply when a device is in the insertion position (FIG. 4) because in the insertion position, the laterally-facing surfaces 68, 70 become radially outwardly-facing surfaces, and the medially-facing surfaces 72, 74 become radially inwardly-facing surfaces.

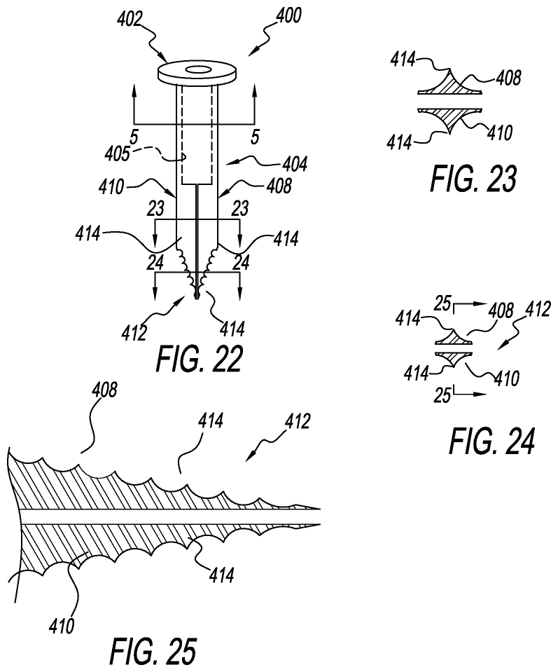

The first alternate embodiment T type tube 400 is shown in FIGS. 22-25. The first alternate embodiment T type tube 400 is constructed generally similarly to the T type tympanostomy tube 30 discussed above in connection with FIGS. 3-10, except for the fact that tube 400 has a serrated distal edge to facilitate the tube 400 incising the tympanic membrane without causing tearing of the membrane. As will be discussed in more detail below, the serrated edges facilitate the sawing through the tympanic membrane, rather than the piercing through of the tympanic membrane. Employing the serrated edges requires certain other modifications of the tube 400 that will be discussed in more detail below.

The first alternate embodiment T type tube 400 includes an enlarged diameter proximal end 402, and a central portion 404. Central portion 404 includes a hollow central passageway 405 that extends generally axially through the central portion 404. The distal portion of the tube 400 includes first and second distal legs 408, 410. As illustrated in FIGS. 23-25, the first and second distal legs 408, 410 are not hollow, but rather solid to enhance their structural rigidity, to better adapt them to piercing through the tympanic membrane. The first and second distal legs 408, 410 each terminate in a pronged end 412 wherein the radius of the pronged end 412 decreases as one moves distally, so that the end has a shape reminiscent of the end of a Phillips screwdriver. The pronged end 412 includes a pair of generally axially extending serrated fins 414 having radially outwardly facing cutting surfaces.

It will be noted that the two distal legs 408, 410 are divided along a plane that extends generally from the distal end of the central portion 404, to the distal end of the pronged end 412. This planar cut results in the pronged end 412 being created from the two end portions of the respective first and second distal legs 408, 410.

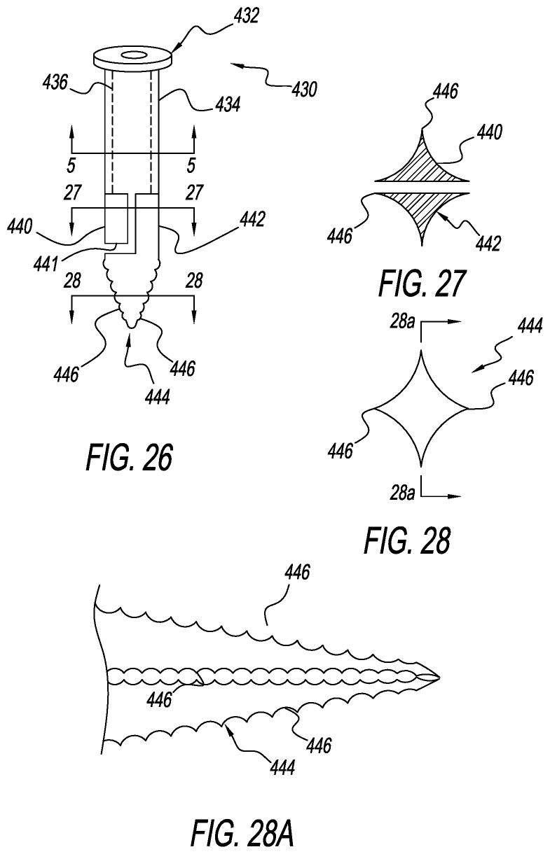

A second alternate embodiment T type tympanostomy tube 430 is shown in FIGS. 26-28A as also including an enlarged diameter proximal end 432, and a central portion 434. Central portion 434 has a hollow, central passageway 436. It will be noted that a section call out is shown on FIG. 26 that suggests that the user view FIG. 5, for the section view taken along FIGS. 5-5. This use of FIG. 5 to illustrate tympanostomy tube 430 indicates that tympanostomy tube 430 is generally identical in this view with tympanostomy tube 30.

First and second distal legs 440, 442 are coupled to the distal portion of the central portion 434. The distal legs 440, 442 extend generally axially when the tympanostomy tube 430 is in its insertion portion, and are movable to extend to about a 90-degree angle when the tympanostomy tube 430 is in its inserted maintenance position.

The first distal leg 440 does not terminate at the distal end of the tympanostomy tube 430. Rather, the first distal leg 440 has a truncated, blunt cut end 441, that terminates the first distal leg 440 at a position just proximal of a four-pronged distal end 444 of the tympanostomy tube 430. This truncated blunt end 441 results in the four-pronged tapered distal end 444 being formed of a single, unitary member, as is illustrated in FIG. 28 and has a shape similar to a Phillips screwdriver with four prongs. The pronged tapered distal end 440 is formed wholly out of the second distal leg 442. The unbroken, tapered pronged distal end 444 is believed by the Applicant to enable the use of a greater number of serrated fins 446, due to the absence of a cut line.

The serrated fins 446 extend generally axially along the tapered outer surface of the tapered, four-pronged distal end 444, and have serrations that extend radially outwardly to form a radially outward extending cutting surface. As with the first alternate embodiment, the plurality of serrated fins 446 promote the cutting of the tympanic membrane by the distal conical end 444 as it incises the tympanic membrane and reduces the likelihood of tearing of the tympanic membrane.

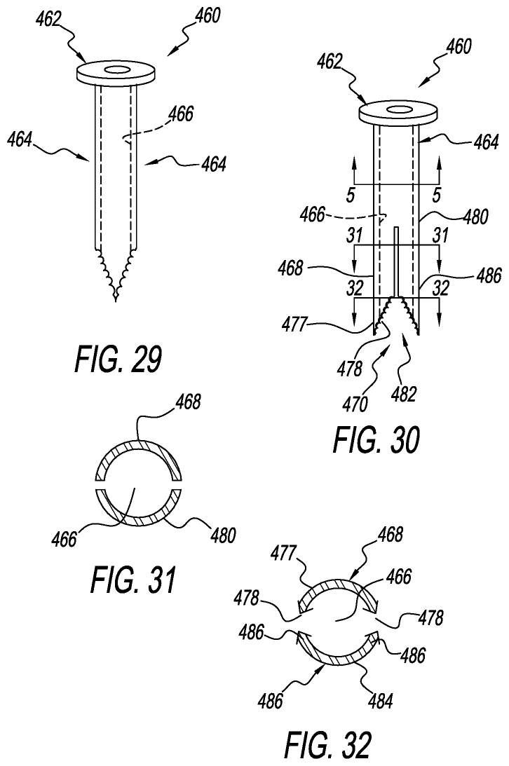

A third alternate embodiment T type tympanostomy tube 460 is shown in FIGS. 29-32. The embodiment shown in FIGS. 29-32 includes an enlarged diameter proximal end 462 and a central portion 464. The central portion 464 includes an axially extending, central passageway 466 that extends all the way from the opening at the proximal end 462 of the tube 460, to the distal end of the tube 460. Unlike the first and second alternate embodiments, the third alternate embodiment 460 does not include a solid distal end.

First and second distal legs 468, 480 respectively, extend distally from the generally distal end of the central portion 464. The first distal leg 468 and second distal leg 480 terminate at the distal cutting portions 470, 482 of the respective first and second distal legs 468, 480. It will be noted that the distal cutting portion 470 of the first distal leg has a generally inverted conical shape, that includes a hemi-cylindrical outer surface 477 and an angled serrated inner surface 478. Similarly, the distal end 482 also includes a generally hemi-cylindrical outer surface 484 and angled, serrated inner portion 486. As with the first and second alternate embodiments, the serrated inner surfaces 478, 486 help to better cut through the meatus of the tympanic membrane, to help reduce tearing of the tympanic membrane.

Generally, in operation and insertion, the first, second and third alternate embodiments operate generally similarly to the manner discussed above in connection with the first embodiment 30 of the T shaped tympanic tube.

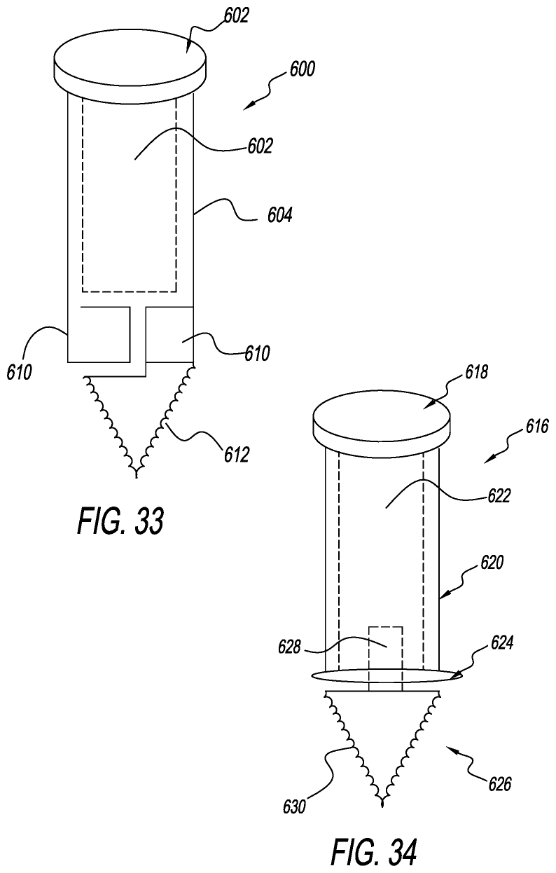

According to additional embodiments, shown in FIGS. 33 and 34, the T shaped tympanic tube may include “built-in” serrated blades. The “T” tympanostomy tube 600 of FIG. 33 generally includes components similar to those of other “T” tympanostomy tubes disclosed herein, such as a proximal end 604, a distal end 610, a central region 604 between the ends 604, 610, a passageway 602, and a flange 602 positioned at the proximal end 604. The “T” tympanostomy tube 600 also includes a serrated blade 612 attached to a limb of the tube.

FIG. 34 includes similar components of a “T” tympanostomy tube 616, e.g., a proximal end 620, a distal end 626, a central region 620 between the ends 620, 626, a passageway 622, and a flange 618 positioned at the proximal end 620. The “T” tympanostomy tube 600 also includes an insert piece that is a detachable serrated “arrowhead” shape that disassociates once deployed in the ear with it falling into the middle ear. It can be made of a resorptive material.

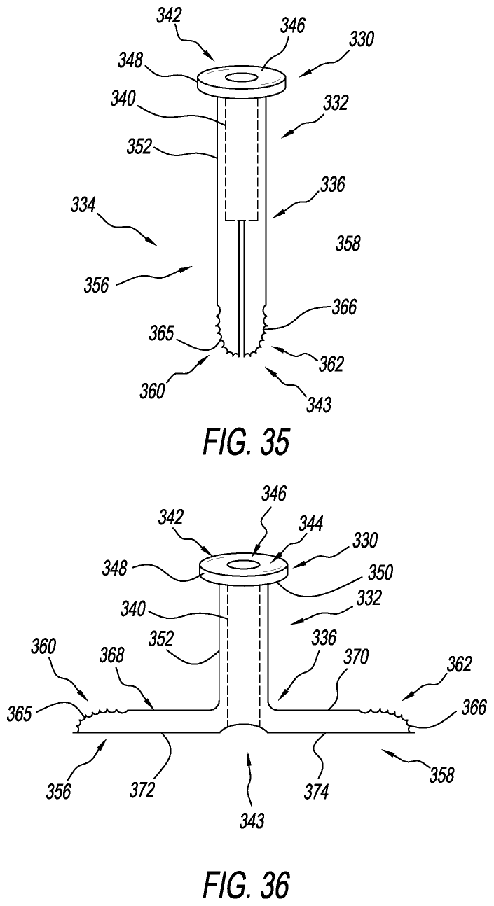

A fourth embodiment “T” tympanostomy tube 330 of the present invention is shown in FIGS. 35-36. The tympanostomy tube 330 includes a proximal portion 332, a distal portion 334, and a central portion 336. The central portion 336 comprises that portion wherein the distal portion 334 joins the proximal portion 332.

An axially-extending passageway 340 extends axially through the proximal portion 332. When the tube 330 is in its insertion position, as shown in FIG. 35, the axially-extending passageway 340 also extends through the distal portion 334. The tube 330 also includes a proximal end 342 that is disposed at the proximal end of the proximal portion 332, and a distal end 343 that is disposed at the distal end of the distal portion 334.

A radially outwardly-extending flange 344 is formed at the proximal end 342 of the ear tube 330. The radially outwardly-extending flange 344 includes an axially proximally-facing end surface 346, and a radially outwardly facing perimetral edge 348. An axially distally-facing surface 350 is disposed in an opposed relationship to the proximally-facing end surface 346.

The radially extending flange 344 is designed to have a diameter larger than the incision made by the tympanostomy tube 330. The purpose of this larger diameter is to ensure that the tympanostomy tube 330 remains in its appropriate place on the exterior surface of the tympanic membrane after insertion. The relatively enlarged diameter flange 344 helps to ensure that the tympanostomy tube is not moved medially out of its engagement with the incision in the tympanic membrane, and through the tympanic membrane into the tympanic cavity.

The proximal portion 332 includes an axially-extending radially outwardly-facing generally cylindrical surface 352, which extends generally from the proximal end to the distal end of the proximal portion 332.

The distal portion 334 includes at least two distally disposed legs including a first distal leg 356 and a second distal leg 358. The first and second distal legs 356, 358 include, respectively, first and second distal ends 360, 362. The first and second distal ends 360, 362 are beveled or otherwise configured to have sharpened edge surfaces, that comprise cutting surfaces. The first and second distal legs 356, 358 should have distal ends 360, 362 that are designed to be sharp enough to easily penetrate the tympanic membrane, upon the exertion of an axially and medially directed force on the tympanostomy tube 330, such as an axially-directed force that is applied to the proximally-facing surface 346 of the proximal flange 342 in a manner to move the tube 330 medially toward the tympanic cavity.

Preferably, the distal ends 360, 362 contain toothed or serrated surfaces 365, 366. The toothed or serrated surfaces 365, 366 are provided for enabling the user to facilitate insertion of the tympanostomy tube 330 into the eardrum by moving the tympanostomy tube in a back and forth direction, to “saw” through the eardrum, rather than to pierce through the eardrum with a sharp point, as is shown by some prior art references. (See Cinberg et al., U.S. Pat. No. 5,254,120).

The Applicant, believes that the use of a serrated edge 365, 366 that is capable of sawing through the tympanic membrane, has significant advantages over the use of a sharp point that is used to pierce through the tympanic membrane. In particular, the Applicant believes that the sawing type action helps to reduce the likelihood of tearing, rupturing and damaging the tympanic membrane, when compared to a pointed device that pushes through by piercing the tympanic membrane.

To understand the advantages of this sawing motion, it is important to understand the nature of a tympanic membrane, and in particular, the mechanical aspects of the tympanic membrane. A tympanic membrane is much like a musical drum head, as it comprises tautly stretched tissue. As the name implies, an eardrum functions somewhat similarly to a drum head. In particular, vibrations caused by sounds that occur within “hearing distance” of the eardrum, cause the eardrum to vibrate. Vibrations of the eardrum transmitted through both fluid and bone structures in the middle and inner ear.

For the tympanic membrane to function properly, it must be tautly stretched so that it is capable of vibrating in response to sound waves that strike it. If the tissue were not stretched tightly but rather was subject to movement and significant expansion and contractions, the vibrations caused by the sound wave would not be transmitted by the eardrum. Rather, the “soft, non-taut tissue” would tend to damp the vibration and not transmit the vibrations along the inner ear part.

The tautness of the eardrum carries with it the benefit of being able to transmit vibrations from the ear canal to the middle ear, and ultimately, through the bones and tissue of the middle and inner ear to the cochlea in which hair-like nerve cells pick up vibrations and transmit the vibrations as neural output to the brain, that can then process the nerve signals into sound. However, this tautness has drawbacks. In particular, because of the tautness of the eardrum, the sharp piercing of the eardrum has a propensity to result in a “stellate” fracture. This stellate fracture is a fracture that is typically characterized by a puncture point results in a plurality of radially extending tears that extend radially outwardly from the puncture point. Another example of a stellate fracture occurs when one sticks a pin in a balloon, to thereby cause the balloon to explode. A stellate fracture is also the kind of fracture that might result if one were to pierce or rupture a tightly stretched drumhead.

Stellate fractures are problematic if they occur in the eardrum because stellate fractures are prone to healing with the skin cyst (cholesteatoma) from the edges of the stellate fracture. If the stellate fracture of the tympanic membrane occurs that causes a cholesteatoma, further surgeries are usually required to remove the cholesteatoma and to repair the eardrum.

By providing sawing surfaces 365, 366 that are provided on the distal end of the eardrum tube 330 of the present invention, the likelihood of such stellate fracture occurring is reduced.

The Applicant believes that the use of a back-and-forth movement to saw through the eardrum with a serrated surface will reduce the likelihood of stellate fractures, when compared to devices that pierce through the eardrum, such as the piercing device disclosed in Cinberg U.S. Pat. No. 5,254,120.

By employing cutting edge containing distal ends 360, 362, the need for using a knife to make a separate incision is thereby obviated. The insertion goes from two steps (i.e., (1) an incision followed by (2) the tube insertion, to one step (i.e., incise and insert, all in one). As best shown in FIGS. 35 and 36, the distal legs 356, 358 are moveable between an insertion position (FIG. 35) and a maintenance position (FIG. 36). In the insertion position (FIG. 35), the distal legs 356, 358 assume a position wherein they are disposed generally co-axially to the long axis of the passageway 340. In the insertion position, the cutting edges 360, 362 are positioned to cut into a membrane, such as the tympanic membrane, upon an axially, medially exerted sawing force upon the tympanostomy tube 330.

From the insertion position (FIG. 35) the distal legs 356, 358 can be moved into a maintenance position as shown in FIG. 36. In the maintenance position, the long axes of each of the first and second distal legs 356, 358 are disposed at least at an oblique angle to the long axis of the passageway 340. Preferably, as shown in FIG. 3, the first and second legs 356, 358 are disposed generally co-axially to each other, and along an axis that is generally perpendicular to the long axis of the passageway 340.

When in the maintenance position as shown in FIG. 36, the distal legs 356, 358 each include laterally (exteriorly) facing surfaces 368, 370 and medially (interiorly) facing surfaces 372, 374. The designations “medial” and “lateral” are used to describe these surfaces because, when in the maintenance position and inserted into an ear, the laterally-facing surfaces 368, 370 face laterally, and are disposed against the medially-facing surface of the eardrum 398.

The medially-facing surfaces 372, 374 face medially inwardly in the middle ear. It will be noted that the medial/lateral designations do not necessarily apply when a device is in the insertion position (FIG. 35) as in the insertion position, the laterally-facing surfaces 368, 370 become radially outwardly-facing surfaces, and the medially-facing surfaces 372, 374 become radially inwardly-facing surfaces.

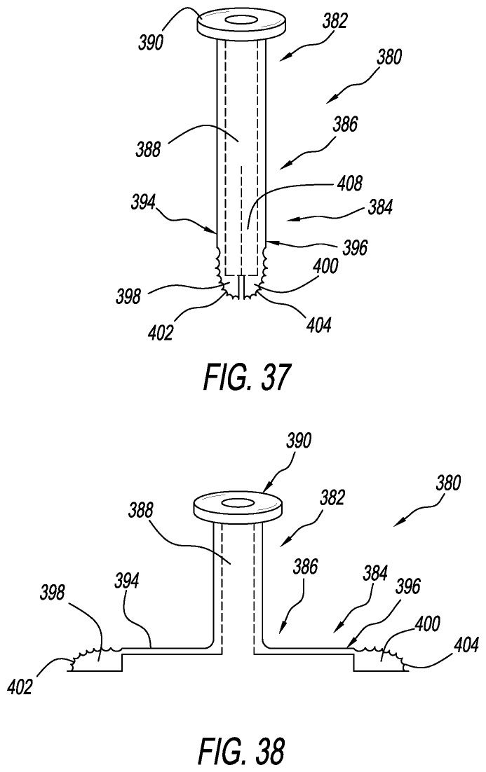

The third embodiment T-type tympanostomy tube 380 of the present invention is shown in FIGS. 37 and 38. Tympanostomy tube 380 includes a proximal portion 382, a distal portion 384 and a central portion 386. An axially extending passageway 388 extends between the proximal and distal ends of the tube 380, and a radially outwardly extending flange 390 is disposed at the proximal end. The distal portion 384 includes a first distal leg 394 and a second distal leg 396. The first distal leg 394 includes a first distal end 398 that includes a first toothed or serrated surface 402. Similarly, the second distal leg 398 includes a second distal end 400 that includes a second toothed or serrated surface 404.

The primary difference between tube 380 and 330 (FIGS. 35 and 36), is that the axially extending passageway 388 also includes a distal portion 408 that extends within the distal portion 384 of the tube 380.

Viewed another way, the “legs” 356, 358 of device 330 are generally solid, whereas the legs 394, 396 of tube 380 are generally hollow. However, the legs 394, 396 terminate at somewhat solid distal ends 398, 340 wherein the toothed surfaces 402, 404 reside.

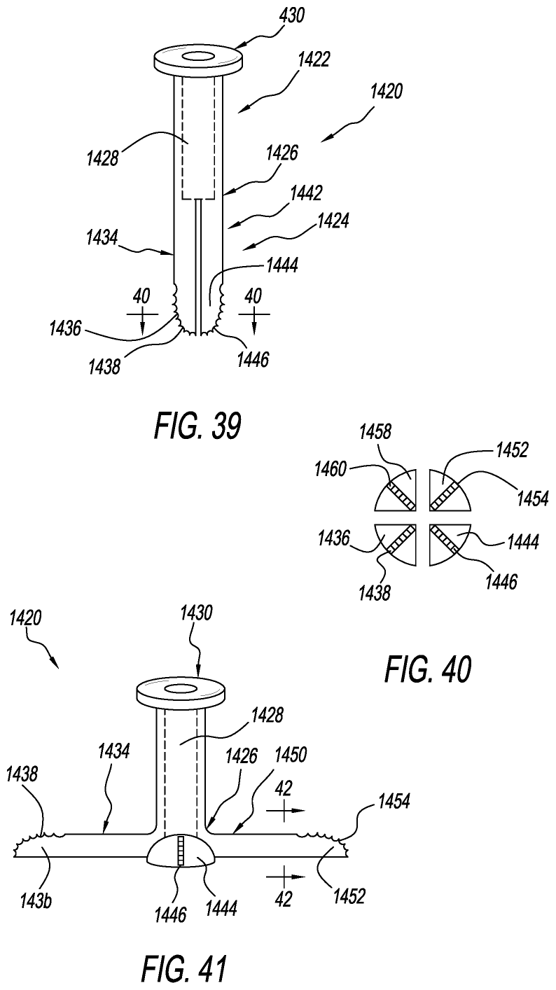

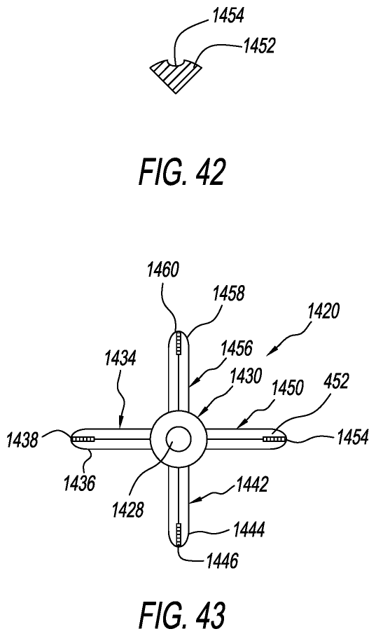

A quad tube embodiment t-type tympanostomy tube 1420 of the present invention is shown in FIGS. 39-43. The quad tube tympanostomy tube 1420 includes a proximal portion 1422, a distal portion 1424 and a central portion 1426 that represents the portion wherein the proximal portion 1422 and distal portion 1424 meet. Additionally, an axially extending passageway 1428 extends through the proximal portion, and a radially outwardly extending flange 1430 is disposed at the proximal end of the proximal portion 1422. In the above respects, quad-legged tympanostomy tube 1420 is generally similar to bi-pod tympanostomy tube 380.

A primary difference between the quad legged tympanostomy tube 1420 and bi-pod tympanostomy tubes 380 or 330 is that the quad legged tympanostomy tube 1420 has four legs including a first distal leg 1434, a second distal leg 1442, a third distal leg 1450, and a fourth distal leg 1456. As shown in FIG. 40, the legs 1434, 1442, 1450, 1456 are each somewhat “pie slice shaped”, so that when in the insertion position, as shown in FIG. 39, a generally cylindrical distal portion 1424 is formed.

The first distal leg 1434 includes a first distal end 1436 having a first toothed (serrated) surface 1438. The toothed surface 1438 extends in generally a proximal distal direction when in the insertion position, to facilitate back and forth movement of the toothed surface 1438 across the tympanic membrane, so that the first toothed surface 1438 (along with its corresponding teethed surfaces 1446, 1454, 1460), can form a generally linear incision in the tympanic membrane.

The second distal leg 1442 includes a second distal end 1444 and a second toothed surface 1446 that is generally similar to the first toothed surface 1438. Additionally, the third and fourth distal legs 1450, 1456 include respective third and fourth distal ends 1452, 1458, that incorporate respective third and fourth toothed (serrated) surfaces 1454, 1460 that are generally similar to the first and second distal ends 1436, 1444 and first and second toothed (serrated) surfaces 1438, 1446.

A benefit of the quad tube is that the use of four splayed legs when in the maintenance position, as shown in FIG. 41, is likely to provide a more stable grip on the interior surface of the tympanic membrane, to thereby be more resistant to the dislodging of the tympanostomy tube 330 from its position in the tympanic membrane when so inserted therein.

It will be noted that when in the insertion position, the toothed surfaces 1454, 1438, and 1460, 1466 of opposed legs are aligned so as to facilitate cutting in a back and forth direction, regardless of whether it is moving in a back and forth direction from legs one and three 1434, 1450 or in a back and forth direction relative to legs two and four 1442 and 1456.

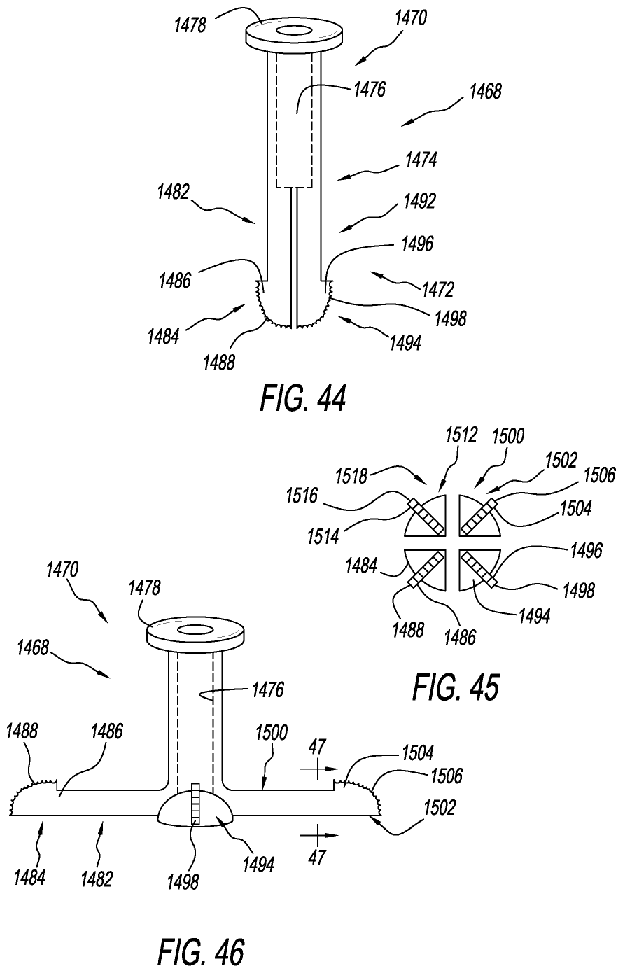

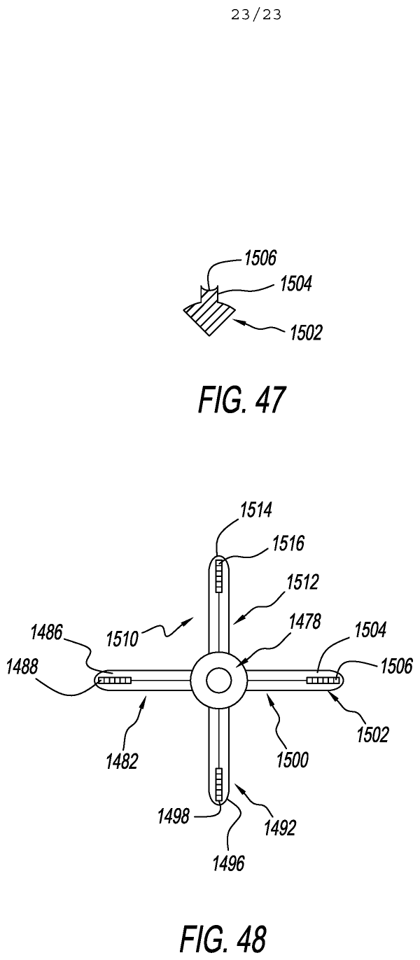

Another quad-legged embodiment T-type tympanostomy tube 1468 is shown in FIG. 44-48. Tympanostomy tube 1468 includes a proximal portion 1470, a distal portion 3 and a central portion 1474. An axially extending passageway 1476 extends between the proximal and distal ends of the tube 1468 and a radially outwardly extending flange 1478 is disposed at the proximal end. The distal portion 1472 includes a first distal leg 1482, a second distal leg 1492, a third distal leg 1500, and a fourth distal leg 1510.

The first distal leg 1482 includes a first distal end 1484 and a raised, blade-like portion 1486 that includes a toothed or serrated surface 1488 on the upper edge thereof. The presence of this raised blade portion 1486 is a primary distinguishing feature between the second embodiment quad legged T-tube 1468 and the first embodiment quad legged T-tube 1420.

Similarly, the second distal leg 1492 includes a second distal end 1494 having a second raised blade-like portion 1496 that includes a second toothed surface 1498. Likewise, the third distal leg and fourth distal leg 1500, 1510 include respective third and fourth distal ends 1502 and 1512, that include respective third and fourth raised blade-like portions 1504, 1514 that include respective third and fourth toothed surfaces 1506, 1516.

It is believed that an advantage of the use of blade portions 1486, 1496, 1504, 1514 is that the raised blade portions 1486, 1496, 1504, 1514 are capable of better positioning the toothed surfaces 1488, 1498, 1506, 1516 on the surface of the tympanic membrane to facilitate the incising of the tympanic membrane caused by the back and forth sawing-like motion of the toothed surfaces 1488, 1498, 1506, 1516 of the tympanostomy tube 1468.

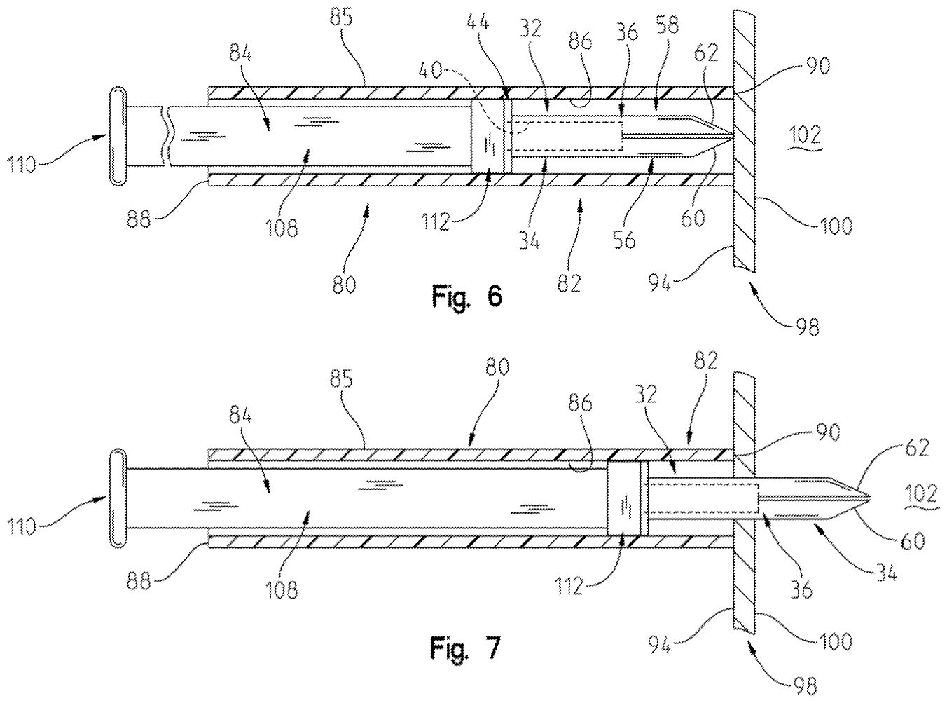

The method and process for inserting the tympanostomy tubes 30, 330, 380, 1470, 1420, 1468 of the present invention is best described to with respect to FIGS. 6-10.

In order to insert the T-type tympanostomy tubes 30, 330, 380, 1420, 1468 into an eardrum, an insertion device 80 (or the alternative insertion device 580 of FIGS. 11-19C) is preferably employed. The insertion device 80, which may also be referenced as a T-tube plunger inserter, includes a guide tube member 82, and a plunger or piston 84. The guide tube member 82 is generally tube-like in configuration, and preferably has a cylindrical radially outwardly-facing exterior surface 85. A generally cylindrical radially inwardly-facing surface 86 defines an axially extending interior passageway 87 (FIG. 9) which extends between the proximal end 88 and the distal end 90 and is open at both the proximal end 88 and the distal end 90. The passageway 87 accommodates the interiorly positioned plunger 84.

The guide tube 82 is sized and positioned so that it can be inserted into the external auditory canal, with the proximal end 88 being disposed exteriorly outwardly of the external auditory canal by a sufficient distance so that it can be grabbed and manipulated by the surgeon. The distal end 90, when the tube 82 is fully inserted, is placed near and/or contacts the laterally (exteriorly) facing surface 94 of the tympanic membrane 98.

The tympanic membrane 98 generally includes a laterally (exteriorly) facing surface 94, and a medially (interiorly) facing surface 100. The laterally facing surface 94 of the tympanic membrane 98 serves as the interior terminus of the external auditory canal, and the medial surface 100 serves as a wall of the tympanic cavity 102. Like a drumhead, the tympanic membrane 98 stretches across the external auditory canal.

A plunger member 84 is provided for axially moving the tympanostomy tube 30 (or 330, 380, 1420 or 1468) in an axially medial direction down the tube 82. The plunger 84 may comprise something as simple as a cylindrical rod. Alternately, the rod or plunger 84 may be formed as a plunger-type mechanism that is constructed similarly to a plunger of a syringe.

One preferred feature of the plunger 84 is that it be sized appropriately. In particular, the plunger 84 should have a flange or head member 110 that has a diameter wider than the interior diameter of the tube 82. This should be done so as to enable the plunger 84 to be inserted only to a certain depth in the tube 82. Preferably, the length of the plunger 84 and the length of the tympanostomy tube 30 should be complementarily sized so that at full insertion of the plunger 84, the tympanostomy tube 30 has been moved axially in a direction and to a point where the tympanostomy tube 30 is appropriately seated within the tympanic membrane 98. The plunger 84 includes a cylindrical body portion 108, a proximal end 110 that includes enlarged diameter head 110, and a distal end 112 (also referred to herein as a forward end of the plunger 84).

Turning now to FIG. 6, the tympanostomy tube 30 and plunger 84 are shown in a position wherein the tube 82 is inserted into the auditory canal, to a point wherein the distal end 90 of the tube 82 rests against the lateral surface 94 of the tympanic membrane 98. The tympanostomy tube 30 is shown in its insertion position wherein the distal legs 56, 58 are disposed at a generally co-axially relationship with the axis of the axially-extending passageway 40.

The plunger has its distal end 112 disposed on and engaged with the upper surface of the radially outwardly extending flange 44 of the tympanostomy tube 200, with the proximal end 110 of the plunger 84 being disposed exteriorly of the tube 82. FIGS. 6 and 7 do not show the length of the plunger 84 at full scale, due to space limitations. Had these space limitations not existed, the head 110 of the plunger would be shown as extending out further from the proximal end 88 of the tube 82.

FIG. 7 is a progressive view that shows that the insertion procedure, using a T-tube plunger inserter, has progressed to the point wherein the plunger 84 has been moved axially medially, to push the tympanostomy tube 30 axially medially. This axially medial (distal) movement of the tympanostomy tube 30 has permitted the cutting edge distal ends 60, 62 to pierce and incise the tympanic membrane 98. In the position shown in FIG. 7, the distal legs 56, 58 are in their insertion position, and the distal ends 56, 58 just barely extend through the tympanic membrane 98, so that only the cutting edges 60, 62 have emerged into the tympanic cavity 102.

In those tympanostomy tubes 330, 380, 1420, 1468 that include toothed surfaces, the distal end of the tympanostomy tubes are preferably moved in a back and forth direction along the line in which the teeth extend to incise the tympanic membrane by sawing through the tympanic membrane. As discussed above, the use of a cutting or sawing motion along the surface of the membrane incises the membrane with a lower risk of creating a stellate fracture, when compared to an incision made by a piercing movement as disclosed by the Cinberg reference cited above.

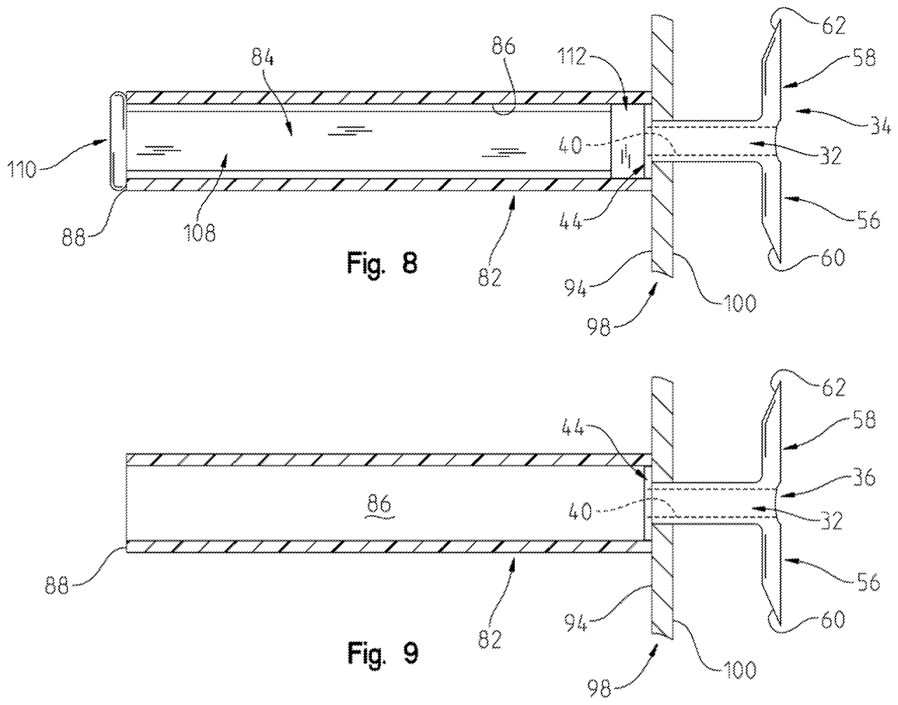

Turning now to FIG. 8, it will be noted that the plunger 84 is fully extended (to its permissible position) into the interior passageway 87 of the tube 82, such that the distal end 112 of the plunger 84 is adjacent to the distal end 90 of the tube 82. Note also that the distal facing surface of the plunger head 110 rests against the proximal end 88 of tube 82. In this position, the radially outwardly extending flange 44 should rest against, or be close to resting against, the lateral surface 94 of the tympanic membrane 98. Additionally, the proximal portion 32 of the tube 30 is positioned so that it is generally co-extensive with, and interiorly-disposed within the tube 82.

The distal portion 34 and much of the central portion 36 of the tympanostomy tube 30 are disposed in the tympanic cavity, so that the laterally-facing surfaces 68, 70 (FIG. 3) of the distal legs 56, 58 are disposed adjacent to, and possibly resting against, the medially facing surface 100 of the tympanic membrane 98. It also should be noted that the distal legs 56, 58 have moved from their insertion position to their maintenance position. In the maintenance position, the first and second distal legs 56, 58 are disposed at an oblique angle to the axis of the central passageway 40, and preferably, are disposed generally perpendicularly to the axis of the central passageway 40.

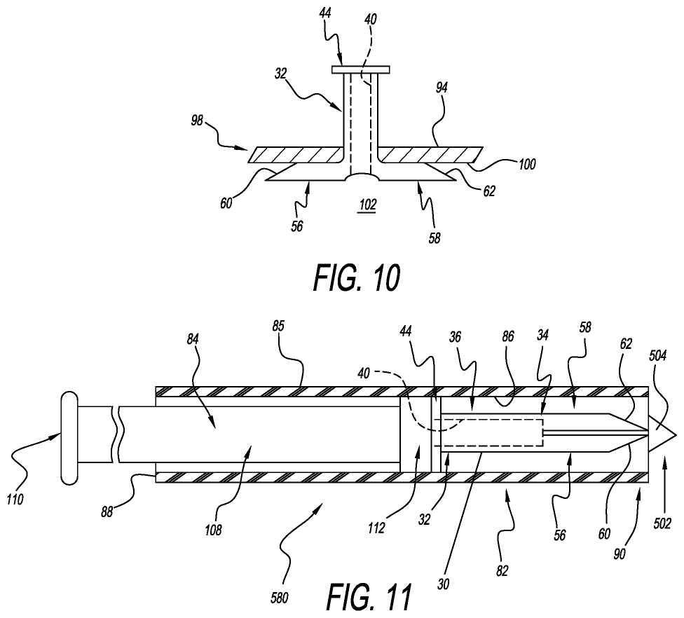

FIG. 9 is similar generally to FIG. 8. However, the plunger 84 is removed. As best shown in FIG. 10, the tube 82 is also removed, and the final resting place of the tympanostomy tube 30 is shown, with the distal legs 56, 58 being moved into their maintenance position. Because of the relatively enlarged radial diameter of the proximal end flange 44, and the relatively enlarged radial diameter of the legs 56, 58, the tympanostomy tube 30 (when in the maintenance position) is prevented from moving axially within the incision in the tympanic membrane 98. The flange 44 and the legs 56, 58 thereby help to prevent the tympanostomy tube 30 from being dislodged from the tympanic membrane 98, either by sliding laterally outwardly or medially inwardly. The axially extending passageway 40 provides a vent tube between the external auditory canal and the tympanic cavity 102, to help prevent the buildup of fluid therein.

Referring now to FIG. 11, an alternative tympanostomy insertion system 580 will now be described. The tympanostomy insertion system 580 includes a guide tube 82, which includes a cutting member 502 located toward the distal end 90 of the guide tube 82. The cutting member 502 can be fixedly coupled and/or integral with the distal end 90 of the guide tube 82.

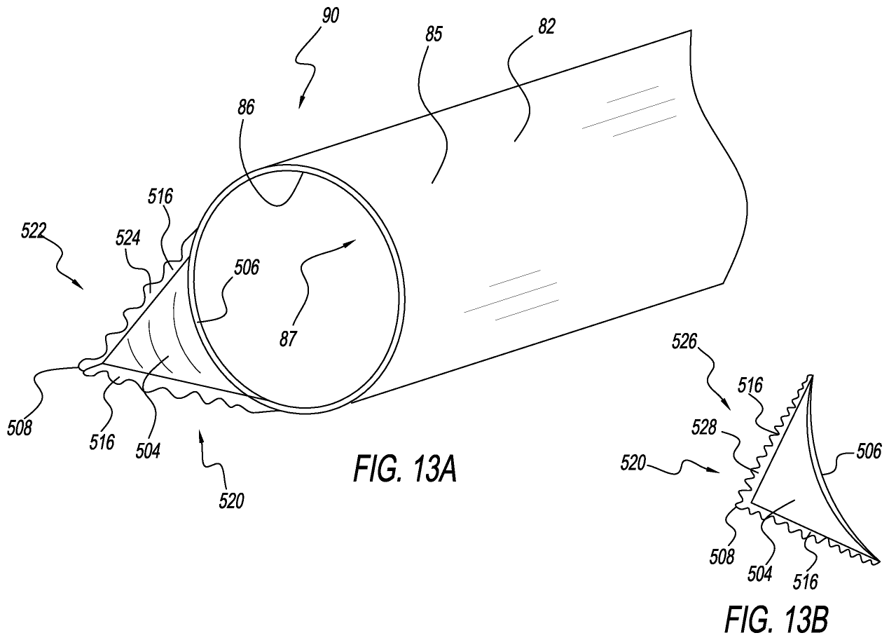

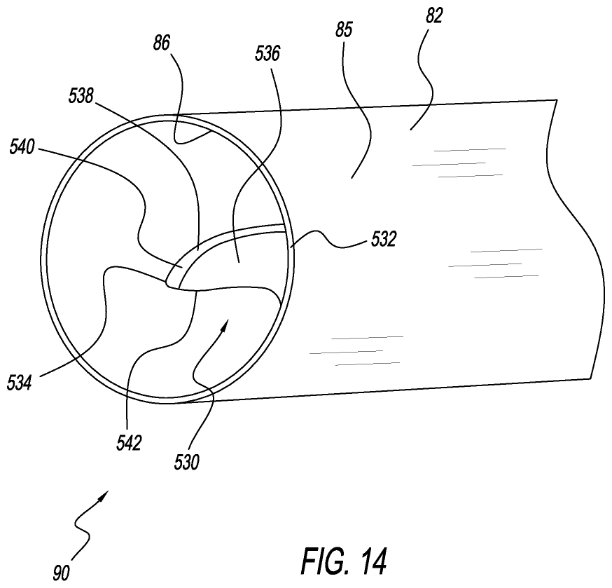

The guide tubes 82 of FIGS. 11 and 15-19C are shown having different cutting members. FIG. 11 is shown as including cutting member 502, which is depicted as taking the form of a bladed cutting member 502; however, the cutting member at the distal end 90 of the guide tube 82 can take a variety of forms, which include, but are not limited to those alternative forms described in connection with FIGS. 13A-14.

Additionally, the tympanostomy insertion device 580 is depicted as including tympanostomy tube 30; however, it is contemplated that the insertion device 580 can include a variety of tympanostomy tubes, such as tympanostomy tubes 330, 380, 430, 460, 1420, 1468, and 1470 as are discussed herein. The tympanostomy tube included in the insertion device 580 is preferably a one-step device, including cutting surfaces on the at least one distal leg thereof.

Viscous fluid within the tympanic cavity 102, such as thick mucus, will not drain through the passageway 40 of the tympanostomy tube 30. Additionally, such viscous fluids often cannot be successfully suctioned through the passageway 40 of the tympanostomy tube 30.

Referring briefly to FIG. 20, it has been discovered that such viscous fluids can be easily suctioned from the tympanic cavity 102 if a vent hole 510 is provisioned into the tympanic membrane 98, in addition to the tympanostomy tube 30. As is illustrated, when a suction device 512 is placed into the vent hole 510, viscous fluid 514 is suctioned outwardly from the tympanic cavity 102 through the suction device 512 located in the vent hole 510. Air 531 can pass through the passageway 40 of the tympanostomy tube 30. It is believed that the provision of airflow into the tympanic cavity 102, through the addition of the vent hole 510 within the tympanic membrane 98, enables the proper suction of the viscous fluid 514. However, the practitioner may alternatively insert the suction device 512 thorough the passageway 40 of the tympanostomy tube 30 (e.g., with viscous fluid 514 being suctioned outwardly from the tympanic cavity 102 through the passageway 40 and with air entering the tympanic cavity 102 through the vent hole 510).

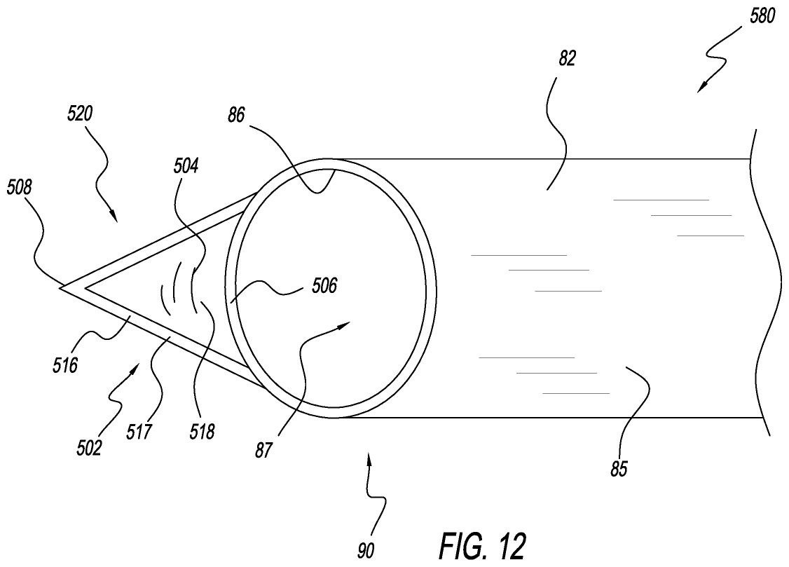

Turning to FIGS. 11-12, the cutting member 502 includes a body 504 which extends between a proximal end 506 and a distal end 508. The cutting member 502 can include an overall triangular shape 520. Opposing outer sides include cutting surfaces 516. The cutting surfaces 516 are sufficiently sharpened to incise the tympanic membrane 98. The cutting surfaces 516 are depicted as being blade-like sharpened surfaces 517.

The body 504 of the cutting member 502 can include a curvature 518 which is substantially the same curvature as the guide tube 82. In this manner, the proximal end 506 of the cutting member can extend along the outer edge of the guide tube 82. The distal end 508 of the cutting member 502 extends outwardly and away from the distal end 90 of the guide tube 82.

FIGS. 13A depicts an alternative cutting member 522 extending outwardly from the guide tube 82. This alternative cutting member 522 is depicted as including serrated tooth-like cutting surfaces 524.

FIG. 13B depicts an alternative cutting member 526 which is depicted as including serrated saw-like cutting surfaces 528. The integration of the serrated surfaces 524 and/or 522 can be advantageous to reduce potential stellate fracturing of the tympanic membrane. Specifically, the use of a sawing-type motion (e.g., back and forth) with a serrated cutting surface has been discovered to greatly reduce the occurrence of stellate fractures during incising of the tympanic membrane.

FIG. 14 depicts yet a further alternative cutting member 530. The cutting member 530 is depicted as taking the form of a scalpel-like member 536 which extends between a proximal end 532 and a distal end 534. The proximal end 532 is fixedly attached with, or integral with, the distal end 90 of the guide tube 82. The scalpel-like member 536 includes a cutting surface 538 located on a first side and non-cutting surface 542 located on a second side, opposite the first side. The cutting surface 538 is depicted as taking the form of blade-like sharpened surface 538; however, it is contemplated that the cutting surface 538 can include serrations.

The cutting member may be described as having an arrowhead-like shape, or wedge-shaped, piercing tip. Numerous different arrowhead-like shapes are known and are contemplated for use in the present disclosure. The arrowhead-like shape may include a triangular or quadrilateral shape and may include external barbs, or projections.

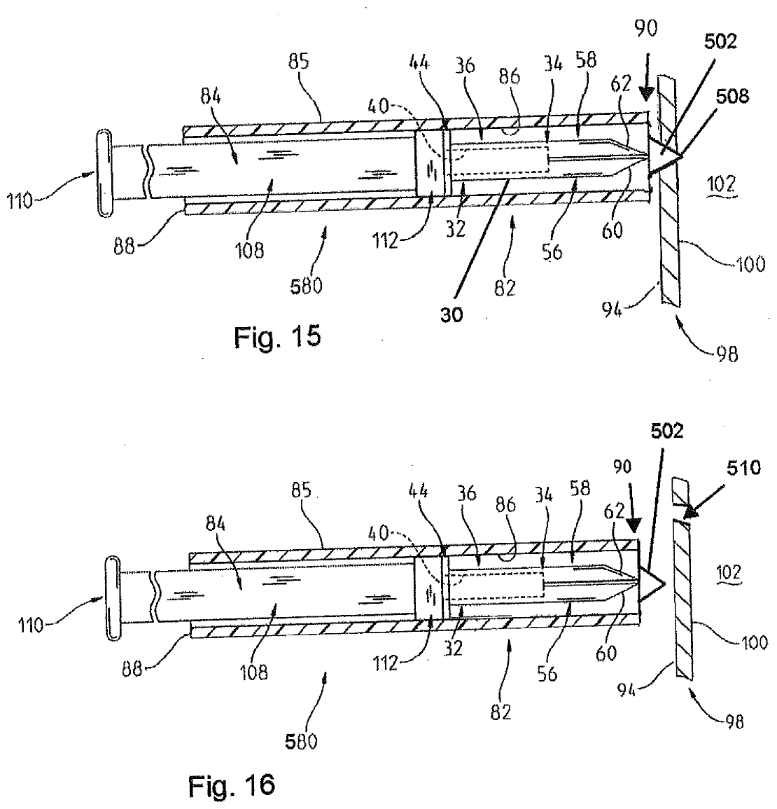

Referring now to FIGS. 15-18, an exemplary use of the insertion device 580, according to the present disclosure will now be described. According to an exemplary procedure, a practitioner can utilize the fixed cutting member 502 to create a first incision, discussed as vent hole 510, and can then utilize the cutting surfaces on the distal legs 60, 62 to create a second incision in the tympanic membrane 98 through which the tympanostomy tube 30 can be inserted.

The practitioner can locate the cutting member 502 at the distal end 90 of the guide tube 82 near the tympanic membrane 98. The practitioner can incise the tympanic membrane 98 at a first location with the cutting member 502 which is fixedly coupled to, or integral with, the guide tube 82. The practitioner will extend the cutting member 502 through the tympanic membrane 98 (shown in FIG. 15) creating an incision 510. As the cutting member 502 is fixedly coupled with the guide tube 82, the practitioner 502 can control the cutting member 502 via the guide tube 82 (e.g., any movement of the guide tube 82 will create movement of the cutting member 502).

Should the cutting member 502 include a blade-like sharpened surface 517, the practitioner may incise the tympanic membrane 98 utilizing a forward motion or a blade like cutting motion. If the cutting member 502 includes a serrated cutting surface, the practitioner may incise the tympanic membrane 98 with a saw-like cutting motion. Specifically, the practitioner can move the serrated cutting member (e.g., cutting members 522, 526) back and forth on the tympanic membrane 98, while the distal end 508 of the cutting member sawingly engages the tympanic membrane 98. As has been discussed herein, use of such saw-like motion has been discovered to significantly reduce the occurrence of stellate fracturing within the tympanic membrane during the incision thereof.

The practitioner can now align the insertion device 580 to create a second incision in the tympanic membrane 98, through which the tympanostomy tube 30 can be inserted. FIG. 16 depicts the guide tube 82 moved slightly outwardly from the tympanic membrane 98, with the distal end 508 of the cutting member 502 removed from the vent hole 510 which was incised in the tympanic membrane 98. The guide tube 82 can be moved to a new location where it is desired to insert the tympanostomy tube 30. FIG. 16 depicts the insertion device 580 moved a small distance downwardly from the location at which the vent hole 510 was created.

When the distal end 90 of the guide tube 82 is oriented toward and positioned sufficiently close to the tympanic membrane 94, the proximal end 110 of the plunger 84 can be depressed which will push the distal legs 60, 62 of the tympanostomy tube 30 outwardly from the distal end 90 of the guide tube 82. When the distal legs 60, 62 are extended outwardly from the guide tube 82, the body 504 of the cutting member 502 is located radially outwardly from the exterior surface of the tympanostomy tube 30.

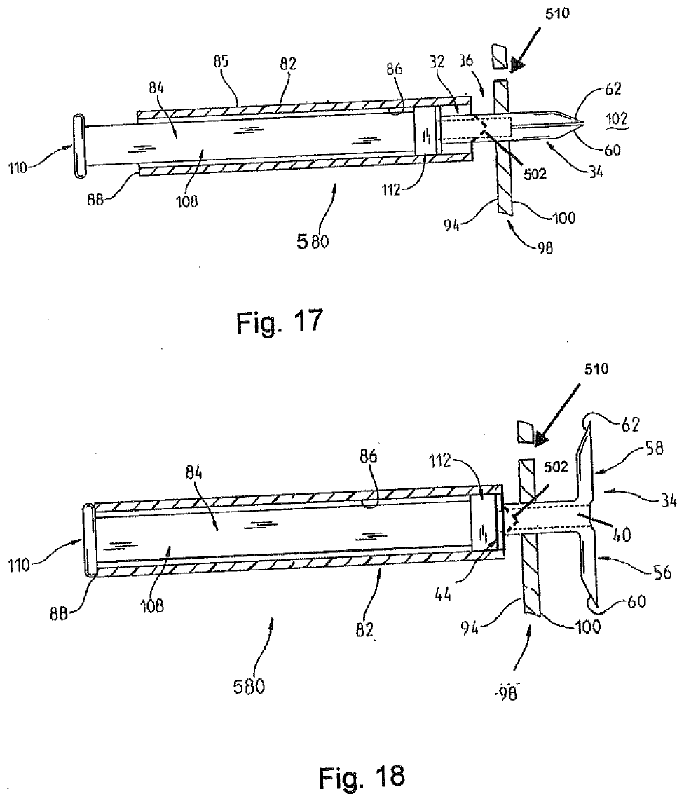

With the cutting surfaces of the distal legs 60, 62 now protruding outwardly from the distal end 90 of the guide tube 82, the practitioner can incise the tympanic membrane 98 with the cutting surfaces of the distal legs 60, 62. The distal legs 60, 62 are inserted through the tympanic membrane 98 and into the tympanic cavity 102, as is shown in FIG. 17. As is illustrated in FIGS. 17-18, the cutting member 502 includes a smaller overall length (e.g., between proximal end 506 and distal end 508) than a length of the tympanostomy tube (e.g., between proximal end 32 and distal end 34).

Depending upon the overall arrangement of the cutting member 502 at the distal end 90 of the guide tube 82, it may be desirable to maintain the distal end 90 of the guide tube 82 a small distance away from the tympanic membrane 94. For example, as depicted in FIG. 17, the cutting member 502 (depicted behind the central portion 36 of the tympanostomy tube 30) is located near, but is not cuttingly engaged with, the tympanic membrane 98 as the tympanostomy tube 30 is inserted therein.

However, it is also contemplated that the cutting member 502 can be disposed on the guide tube 82 sufficiently close to the tympanostomy tube 30 such that the distal end 508 of the cutting member 502 will substantially extend into the same opening as is formed by the cutting surfaces of the distal legs 60, 62. It is also contemplated that the distal end 508 of the cutting member 502 can extend radially inward relative the proximal end 506 (e.g., the pointed distal end 508 can be directed toward and located adjacent the tympanostomy tube 30). It is believed that a close relationship between the distal end 508 of the cutting member 502 and the tympanostomy tube 30 can enable a practitioner to contact the distal end 90 of the insertion device 580 against the tympanic membrane 98 with the distal end 508 of the cutting member 502 extending into the incision created by the cutting surfaces of the distal legs 60, 62.

FIG. 18 depicts the tympanostomy tube 30 after insertion through the tympanic membrane 98, with the tympanostomy tube 30 placed in a maintenance position. As has been discussed herein, with the tympanostomy tube 30 properly inserted through the tympanic membrane 98 and disposed in the maintenance position (e.g., with the legs 60, 62 extended approximately perpendicular relative the central section 36), the plunger 84 can be pulled rearwardly and the insertion device 580 can be removed from the auditory canal.

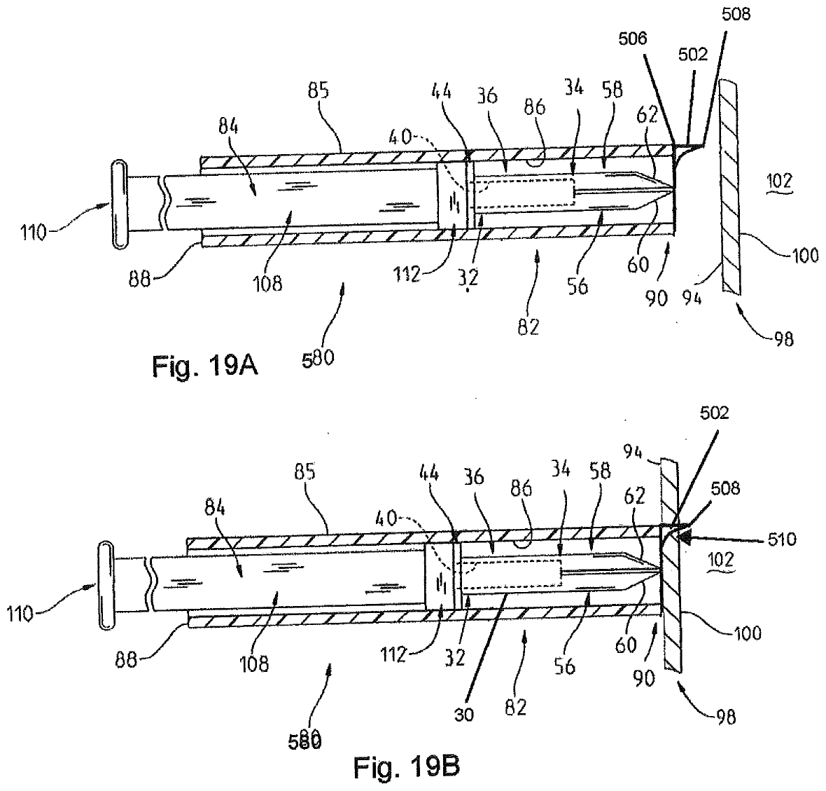

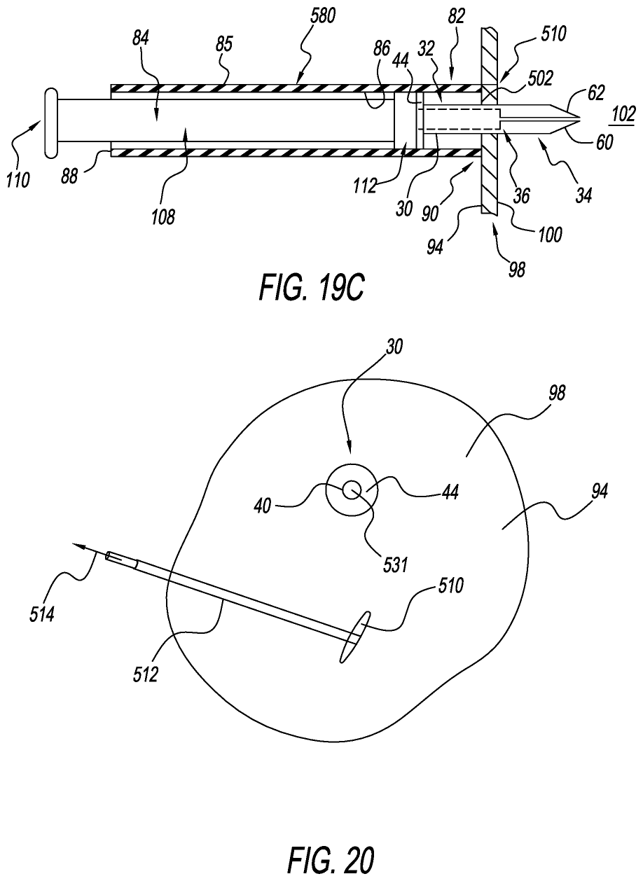

FIGS. 19A-19C depict the insertion device 580 utilized in a one-step fashion. Referring to FIG. 19A, the distal end 508 of the cutting member 502 is positioned near the tympanic membrane 98. The practitioner can then incise the tympanic membrane with the cutting member 502 with either a slicing, piercing, and/or sawing motion, depending upon the cutting member 502 utilized and the practitioners' preferences. FIG. 19B depicts the insertion device 580 located with the distal end 508 of the cutting member 502 extending into the tympanic membrane 98 and with the distal end 90 of the guide tube 82 contacting the tympanic membrane 98.

The practitioner can then push the plunger 84 forward so that the cutting surfaces of the distal legs 60, 62 piercingly incise the tympanic membrane 98 and extend through the tympanic membrane 98, as is best illustrated in FIG. 19C. This exemplary use is referred to as a one-step fashion in that once the practitioner has pierced the tympanic membrane 98 with the cutting member 502, the practitioner can continue the insertion of the tympanostomy tube 30 without the need to reorient the insertion device 580 within the auditory canal.

As can best be seen in FIG. 19C, the vent hole 510 created by the cutting member 502 is located a distance away from the incision created by the tympanostomy tube 30. The cutting member 502 can be positioned at the distal end 90 of the guide tube 82 such that the distal end 508 of the cutting member 508 is positioned radially outwardly relative the outer surface 85 of the guide tube 82 (e.g., to provide further separation between the cutting member 502 and the incision created by the tympanostomy tube 30).

Although specific exemplary uses of the insertion device 580 have been discussed herein, it is contemplated that a skilled practitioner can utilize the insertion device 580 in a variety of manners to successfully insert a tympanostomy tube through the tympanic membrane 98, and to enable the suction of mucus from the tympanic cavity 102 if desired. For example, the practitioner may desire to first insert the tympanostomy tube into the tympanic membrane 98. The practitioner may then utilize the cutting member 502 to incise the tympanic membrane 98 to create a vent hole 510. The practitioner may prefer to create a small incision with the cutting member (e.g., utilizing a saw-like motion) as a pilot hole for the sharpened surfaces of the tympanic member to extend through; however, the creation of such a pilot hole is believed to likely be redundant and unnecessary.

While the invention has been described in connection with what is presently considered to be the most practical and preferred embodiment, it is to be understood that the invention is not to be limited to the disclosed embodiment(s), but on the contrary, is intended to cover various modifications and equivalent arrangements included within the spirit and scope of the appended claims, which scope is to be accorded the broadest interpretation so as to encompass all such modifications and equivalent structures as permitted under the law.

It should be understood that while the use of the word preferable, preferably, or preferred in the description above indicates that feature so described may be more desirable, it nonetheless may not be necessary and any embodiment lacking the same may be contemplated as within the scope of the invention, that scope being defined by the claims that follow.

In reading the claims it is intended that when words such as “a,” “an,” “at least one” and “at least a portion” are used, there is no intention to limit the claim to only one item unless specifically stated to the contrary in the claim. Further, when the language “at least a portion” and/or “a portion” is used the item may include a portion and/or the entire item unless specifically stated to the contrary.

What is claimed is:

1. A tympanostomy tube insertion system, including:
a guide tube including a hollow passageway, the guide tube extending between a proximal end and a distal end;
a first cutting member located at the distal end of the guide tube, the first cutting member configured to incise a tympanic membrane;
a plunger member having a forward end which extends into the hollow passageway at the proximal end of the guide tube; and
a tympanostomy tube disposed within the hollow passageway toward the distal end of the guide tube.

2. The insertion system of claim 1, wherein the tympanostomy tube includes a body extending between a first end and a second end, wherein a second cutting member is disposed at the second end, and wherein the second cutting member is configured to incise the tympanic membrane during insertion of the tympanostomy tube in the tympanic membrane.

3. The insertion system of claim 1, wherein the first cutting member is fixedly coupled with the distal end of the guide tube, and wherein the position of the first cutting member remains fixed relative the distal end of the guide tube.

4. The insertion system of claim 2, wherein the first cutting member is configured to incise the tympanic membrane to create a vent hole, and wherein the second cutting member is configured to incise an aperture in the tympanic membrane to receive the tympanostomy tube.

5. The insertion system of claim 2, wherein the tympanostomy tube includes a first leg and a second leg disposed at the second end, and wherein the second cutting member is located on the first leg, and wherein a third cutting member is located on the second leg.

6. The insertion system of claim 3, wherein the plunger member is configured to act on the first end of the tympanostomy tube, and wherein the second cutting member is configured to extend outwardly from the distal end of the guide tube.

7. The insertion system of claim 1, wherein the first cutting member includes a bladed surface.

8. The insertion system of claim 1, wherein the first cutting member includes a serrated surface.

9. The insertion system of claim 8, wherein the first cutting member is configured to incise the tympanic membrane utilizing a saw-like motion, thereby preventing stellate fracturing within the tympanic membrane.

10. The insertion system of claim 1, wherein the first cutting member includes a scalpel-like member.

11. The insertion system of claim 1, wherein the first cutting member is integral with the distal end of the guide tube.

12. The insertion system of claim 4, wherein a suction device is placed into the vent hole and is configured to suction a fluid outwardly from the tympanic cavity through the suction device located in the vent hole.

13. The insertion system of claim 5, wherein the first and second legs are solid.

14. The insertion system of claim 5, wherein the first and second legs are hollow.

15. The insertion system of claim 2, wherein the tympanostomy tube includes a first leg, a second leg, a third leg, and a fourth leg.

16. The insertion system of claim 15, wherein the first, second, third, and fourth legs include respective second, third, fourth, and fifth cutting members.
